(19) United States
(12) Patent Application Publication
(10) Pub. No.: US 2010/0094075 A1
(43) Pub. Date: Apr. 15, 2010

(54) Expandable medical devices with reinforced elastomeric members and methods employing the same

(75) Inventor: Joseph L. Mark, (US)

Correspondence Address: BINGHAM MCCUTCHEN LLP, 2020 K Street, N.W., Intellectual Property Department, WASHINGTON, DC, 20006, US

(73) Assignee: Hologic Inc.

(21) Appl. No.: 12/285,662

(22) Filed: Oct. 10, 2008

(51) Int. Cl. A61M 36/04

(52) U.S. Cl. 600/3

(57) ABSTRACT

Expandable devices for placement within a patient comprising an elastomeric member in contact with at least one fiber that provide non-differential expansion. Brachytherapy apparatus and methods for performing brachytherapy employing expandable devices comprising an elastomeric member in contact with at least one fiber that provide non-differential expansion.

### BACKGROUND OF THE INVENTION

This invention relates generally to expandable medical devices with elastomeric members, such as balloons, in contact with at least one fiber and apparatus and methods employing the same.

Treatment of Medical Disorders Using Expandable Medical Devices

Medical balloons are one type of expandable medical device that are widely-used in a number of medical procedures. Typically, an uninflated medical balloon is inserted into a space within the patient's body. When the medical balloon is inflated, the volume of the medical balloon expands, and the space is similarly expanded. In procedures such as angioplasty, the medical balloon may be used to open a collapsed or blocked artery.

Medical balloons are often employed with catheters, with or without stents, to treat strictures, stenoses, and/or narrowings in various parts of the human body. Devices with varying designs have been utilized for angioplasty, including stents and grafts or combination stent/grafts.

Procedures involving balloon catheters include percutaneous transluminal angioplasty (“PTA”) and percutaneous transluminal coronary angioplasty (“PTCA”), which may be used to reduce arterial build-up, such as that caused by the accumulation of atherosclerotic plaque. In those procedures, a balloon catheter is typically passed over a guidewire to a stenosis with the aid of a guide catheter. The guidewire extends from a remote incision to the site of the stenosis, and typically across the lesion. The balloon catheter is passed over the guidewire, and ultimately positioned across the lesion.

Once the balloon catheter is positioned appropriately across the lesion, often with fluoroscopic guidance, the balloon is inflated. As a result, the plaque of the stenosis is broken and the arterial cross section is increased. The balloon is then deflated and withdrawn over the guidewire into the guide catheter, and removed from the patient's body of the patient.

Medical balloons are often made of rubber or another compliant material. To inflate such balloons, pressure is increased within the balloon, causing the compliant material to stretch. As the pressure is increased, the balloon expands until it ruptures. Typical medical balloons rupture at approximately 7-20 atmospheres, or about 100-300 psi.

Controlling the dimensions of an expanded medical balloon is difficult. The pressure introduced into the balloon must be sufficient to expand the medical balloon to a therapeutically-effective size, yet insufficient to rupture the balloon. If the balloon expands too much, the treatment site may be stressed and may even be damaged. Rupture of the balloon can have serious negative complications.

Medical balloons are often designed with close tolerances so that the expansion pressure is predictable. Yet, variations in the materials from which the balloon is constructed may lead to under-expansion and/or over-expansion. The equipment used to expand and control pressure within the balloon must be carefully calibrated in an effort to deliver the desired expansion with minimal deviation.

Medical balloons are often used in medical procedures such as angioplasty, orthopaedics, brachytherapy, and the like. In such procedures, it is necessary to force a space within the patient's body.

It is desirable for medical devices, such as medical balloons, not to expand beyond a predetermined size and to maintain a predetermined shape. If a medical device does not expand beyond the predetermined size and maintains its predetermined shape, it is less likely to rupture during the medical procedure.

Treatment of Proliferative Disorders Using Expandable Medical Devices Treatment of proliferative disorders (disorders including or characterized by rapid or abnormal cell growth or proliferation, including tumors, restenosis, abnormal angiogenesis, hyperplasia, and the like) has become increasingly sophisticated in recent years, and improvements in surgical, chemotherapeutic, and brachytherapeutic techniques have led to better outcomes in patients suffering from such disorders. Malignant tumors are often treated by removing as much of the tumor as possible with surgical resection. Yet, the therapeutic value of this procedure is reduced if tumor cells infiltrate into normal tissue surrounding the tumor. To combat this, surgical resection is often supplemented with radiation therapy whereby the residual tumor margin is targeted after resection.

The supplemental radiation therapy is administered through any number of methods, ranging from external beam radiation, stereotactic radiosurgery, and permanent or temporary brachytherapy. “Brachytherapy” refers to radiation therapy delivered by a spatially-confined source of therapeutic rays inserted into a mammalian body at or near a tumor or other proliferative tissue disease site. Due to the proximity of the radiation source, brachytherapy offers the advantage of delivering a more localized dose to the target tissue region. For example, brachytherapy can be performed by implanting radiation sources directly into the tissue to be treated. Brachytherapy is most appropriate where: (1) malignant tumor regrowth occurs locally, within 2 or 3 cm of the original boundary of the primary tumor site; (2) radiation therapy is a proven treatment for controlling the growth of the malignant tumor; and (3) there is a radiation dose-response relationship for the malignant tumor, but the dose that can be given safely with conventional external beam radiotherapy is limited by the tolerance or normal tissue. In brachytherapy, radiation doses are highest in close proximity to the radiotherapeutic source, providing a high tumor dose while sparing surrounding normal tissue. Brachytherapy is useful for treating malignant brain and breast tumors, among others.

Interstitial brachytherapy is often carried out using radioactive seeds, such as ¹²⁵I seeds. Unfortunately, these seeds produce variable dose distributions. To achieve a minimum prescribed dosage throughout a target region of tissue, high activity seeds are often used. This often results in very high radiation doses being delivered to regions closest to the seed(s). That, in turn, often leads to radionecrosis in healthy tissue.

Prior art brachytherapy devices have provided a number of advancements in the delivery of radiation to target tissue. For example, Williams U.S. Pat. No. 5,429,582 (“Williams”), incorporated herein in its entirety for all purposes, describes a method and apparatus for treating tissue surrounding a surgically-excised tumor with radioactive emissions to kill any cancer cells that may be present in the tissue surrounding the excised tumor. To deliver the radioactive emissions, Williams provides a catheter having an inflatable balloon, such as those discussed above, at its distal end that defines a distensible reservoir. After the tumor is surgically removed, the surgeon introduces the balloon catheter into the surgically-created pocket where the tumor had resided. The balloon is then inflated by injecting a fluid having one or more radionuclides into the distensible reservoir via a lumen in the catheter.

The apparatus described in Williams solved some of the problems found when using radioactive seeds for interstitial brachytherapy, but left some problems unresolved. The absorbed dose rate at a target point exterior to a radioactive source is inversely proportional to the square of the distance between the radiation source and the target point. As a result, where the radioactive source has sufficient activity to deliver a prescribed dose, e.g., two centimeters into the target tissue, the tissue directly adjacent the wall of the distensible reservoir, where the distance to the radioactive source is very small, may still be overly “hot” to the point where healthy tissue necrosis may result. Generally, the amount of radiation desired by the physician is a certain minimum amount that is delivered to a region up to about two centimeters away from the wall of the excised tumor. It is desirable to keep the radiation that is delivered to the tissue in the target treatment region within a narrow absorbed dose range to prevent over-exposure to tissue at or near the reservoir wall, while still delivering the minimum prescribed dose at the maximum prescribed distance from the reservoir wall.

U.S. Pat. No. 6,413,204 to Winkler et al., incorporated herein in its entirety for all purposes, provides an apparatus that delivers radiation from a radioactive source to target tissue within the human body with a desired intensity and at a predetermined distance from the radiation source, without over-exposure of body tissues disposed between the radiation source and the target. The apparatus includes a catheter body member having a proximal end and distal end, an inner spatial volume disposed proximate to the distal end of the catheter body member, an outer spatial volume defined by an expandable surface element, such as a balloon, disposed proximate to the distal end of the body member in a surrounding relation to the inner spatial volume, and a radiation source disposed in the inner spatial volume. The inner and outer spatial volumes are configured to provide an absorbed dose within a predetermined range throughout a target tissue. The target tissue is located between the outer spatial volume expandable surface and a minimum distance outward from the outer spatial volume expandable surface. The predetermined dose range is defined as being between a minimum prescribed absorbed dose for delivering therapeutic effects to tissue that may include cancer cells, and a maximum prescribed absorbed dose above which healthy tissue necrosis may result.

In years past, Brachytherapy often calculated the desired radiation dose based on the characteristics of the brachytherapy applicator (device), the radiation source, and the surrounding tissue. Yet, the actual dose delivered was not tested to assure that over- and/or under-treatment did not occur. For example, if the radiation source is a radioactive seed positioned in the center of an expanded balloon, the calculated dose is based on the central positioning of the radiation source. If for some reason the radioactive seed was positioned off center, prior art brachytherapy devices had no means to determine that this harmful situation was occurring. Prior art brachytherapy devices also lacked the ability to directly sense the surrounding tissue and determine the effectiveness of the proliferative tissue disorder treatment. The implantable radiotherapy/brachytherapy radiation-detecting apparatus and methods described in U.S. Pat. No. 7,354,391 to Stubbs, incorporated herein in its entirety for all purposes, remedied that situation by offering a means to deliver and monitor radioactive emissions applied within a mammalian body. There, the device employed included a catheter body member having a proximal end, a distal end, and an outer spatial volume disposed proximate to the distal end of the body member. A radiation source was preferably positioned in the outer spatial volume, and a treatment feedback sensor was disposed on the device.

U.S. Pat. No. 6,482,142 to Winkler et al. (“the '142 Patent”), incorporated herein in its entirety for all purposes, provides brachytherapy apparatus for delivering radioactive emissions in an asymmetric fashion to target tissue surrounding a surgical extraction site. The apparatus includes an expandable outer surface element defining an apparatus spatial volume, a radiation source disposed within the apparatus volume, and a means for providing predetermined asymmetric isodose profile within the target tissue. The brachytherapy apparatus of the '142 Patent include an expandable outer surface defining a three-dimensional apparatus volume configured to fill an interstitial void created by the surgical extraction of diseased tissue and define an inner boundary of the target tissue being treated and a radiation source disposed completely within the expandable outer surface and located so as to be spaced apart from the apparatus volume, the radiation source further being asymmetrically located and arranged within the expandable surface to provide predetermined asymmetric isodose curves with respect to the apparatus volume. The brachytherapy apparatus of the '142 Patent may include an asymmetric radiation shield spaced apart from the radiation source that provides predetermined asymmetric isodose curves with respect to the apparatus volume.

The '142 Patent also provides surgical apparatus for providing radiation treatment to target tissue including an expandable outer surface defining an apparatus volume and a radiation source replaceably disposable within the expandable outer surface, the radiation source comprising a plurality of solid radiation sources arranged to provide predetermined asymmetric isodose curves within the target tissue. The plurality of solid radiation sources may be spaced apart on a single elongate member shaped to provide asymmetric placement of the spaced apart solid radiation sources with respect to a longitudinal axis through the apparatus volume, or may be provided on at least two elongate members extending into the apparatus volume, at least one of the elongate members being shaped to provide asymmetric placement of a radiation source with respect to a longitudinal axis through the apparatus volume. In the surgical apparatus, at least one of the plurality of solid radiation sources may have a different specific activity from at least one other solid radiation source.

U.S. Pat. No. 5,913,813 to Williams et al. (“the '813 Patent”), incorporated herein in its entirety for all purposes, discloses apparatus for delivering radioactive emissions to a body location within a uniform radiation profile, by delivering a desired radiation dose at a predetermined radial distance from a source of radioactivity by providing a first spatial volume at the distal end of a catheter and a second spatial volume defined by a surrounding of the first spatial volume by a polymeric film wall where the distance from the spatial volume and the wall is maintained substantially constant over their entire surfaces. In those apparatus, one of the inner and outer volumes is filled with either a fluid or a solid containing a radionuclide(s) while the other of the two volumes is made to contain either a low radiation absorbing material, e.g., air or even a more absorptive material, such as an x-ray contrast fluid. Where the radioactive material comprises the core, the surrounding radiation absorbing material serves to control the radial profile of the radioactive emissions from the particular one of the inner and outer volumes containing the radionuclide(s) so as to provide a more radially uniform radiation dosage in a predetermined volume surrounding the outer chamber. Where the core contains the absorbent material, the radial depth of penetration of the radiation can be tailored by controlling the core size.

While expandable medical devices, such as medical balloons, continue to offer great advantages in treating a number of mammalian ailments, such as those exemplified above, employment of the expandable members brings with it certain disadvantages. Included within the disadvantages is that upon expansion, the medical device may not be symmetric. For example, the device is either too tight or too loose to administer efficient therapy. Efficient therapy requires non-differential expansion at least because it provides for predictable therapy.

Accordingly, there is a need for an expandable medical device that provides for non-differential expansion.

### SUMMARY OF THE INVENTION

The present invention provides expandable medical devices that are characterized by non-differential expansion during use in the treatment of medical disorders, brachytherapy apparatus employing such devices, and methods for performing brachytherapy employing such devices.

In one embodiment, the expandable device for placement within a patient includes an elastomeric member in contact with at least one fiber that forms a pattern and is oriented in at least two directions within said pattern.

In another embodiment, the expandable device may be employed in a brachytherapy apparatus for delivering radioactive emissions to mammalian tissue. The brachytherapy apparatus includes an elastomeric member for placement within a patient in contact with at least one fiber, a catheter having a proximal end, a distal end, and a spatial volume at the distal end, wherein the spatial volume is defined by the elastomeric member, and a radiation source position disposed in the spatial volume.

In another embodiment, the expandable device may be employed in a method for performing a brachytherapy procedure on a patient. The method includes: inserting into the patient in need of brachytherapy a catheter having a proximal end, a distal end, and a spatial volume at the distal end, wherein the spatial volume is defined by an elastomeric member and at least one fiber in contact with the elastomeric member; inflating or expanding the elastomeric member; inserting a radiation source in the spatial volume of the catheter; and removing the radiation source and the catheter from the patient.

### BRIEF DESCRIPTION OF THE DRAWINGS

The invention will be more fully understood from the following detailed description taken in conjunction with the accompanying drawings:

FIG. 1 illustrates differential expansion often encountered with conventional elastomeric members.

FIG. 2 illustrates an elastomeric member in contact with at least one fiber oriented in two directions located inside the elastomeric member.

FIG. 3 illustrates an elastomeric member in contact with at least one fiber oriented in two directions located outside the elastomeric member.

FIG. 4 illustrates an elastomeric member in contact with at least one fiber oriented in two directions located within said elastomeric member.

FIG. 5 illustrates a brachytherapy apparatus of the present invention for delivering radioactive emissions to mammalian tissue.

### DETAILED DESCRIPTION OF THE PREFERRED EMBODIMENTS

The present invention provides devices, apparatus, and methods for use in treating medical disorders, and more particularly to devices, apparatus, and methods for the treatment of such disorders in mammals by employing elastomeric members in contact with at least one fiber.

As used herein, the term “elastomeric member” includes any distensible device constructed of elastic material, such as a medical balloon. Exemplary elastomeric members include the variety of distensible devices designed for use with surgical catheters. The elastomeric member may be, for example, a balloon, a brachytherapy balloon, or an angioplasty balloon. The elastomeric member may be used, for example, to open and/or clear one or more strictures within a patient.

The elastomeric member may take any configuration that achieves the desired therapeutic result, including, for example, round, square, rectangular, or oval configurations. Expansion of the elastomeric member may occur by any means, including solid, liquid, and/or gas expansion.

The elastomeric member may be permeable, impermeable, and/or semi-permeable to fluid, depending on the needs of the therapy. For example, an elastomeric member may be constructed of a solid material that is substantially impermeable to active components of a treatment fluid (e.g., radiation source material) with which it can be filled, and is also impermeable to body fluids (e.g., blood, cerebrospinal fluid). An impermeable elastomeric member is useful in conjunction with a radioactive treatment fluid to prevent the radioactive material from escaping the treatment device and contaminating the therapeutic site or tissues of the patient.

Alternatively, an elastomeric member may be constructed such that it is permeable to a treatment agent, permitting a treatment agent to pass out of the member and into, for example, a body lumen, body cavity, or therapeutic site. Permeable elastomeric members are useful when the treatment agent is a drug, such as a chemotherapeutic drug which must contact tissue to be effective.

Treatment agents may also be delivered from the surface of an elastomeric member to the surrounding tissue.

Generally, it is preferable that the elastomeric member has a shape that corresponds to the body cavity or site in which it is to be employed. For example, a generally spherical cavity can be filled with a substantially spherical member, whereas an elongated member is suitable for an elongated cavity, such as a blood vessel. Irregular member shapes may also be appropriate, depending on the needs of the therapy.

In certain embodiments, the elastomeric member is selected such that upon inflation or expansion the member does not compress the tissue which is being treated nor the surrounding tissue. For example, in one embodiment, when the elastomeric member is placed within a cavity left by surgical removal of tissue, the member is not inflated or expanded to a size substantially larger than the size of the cavity. However, in certain other embodiments, the elastomeric member is inflated or expanded so as to compress tissue. For example, when the proliferative disorder being treated is restenosis of a blood vessel, the member is inflated or expanded to a size large enough to compress the excess tissue, and may also provide chemotherapy, brachytherapy, or the like.

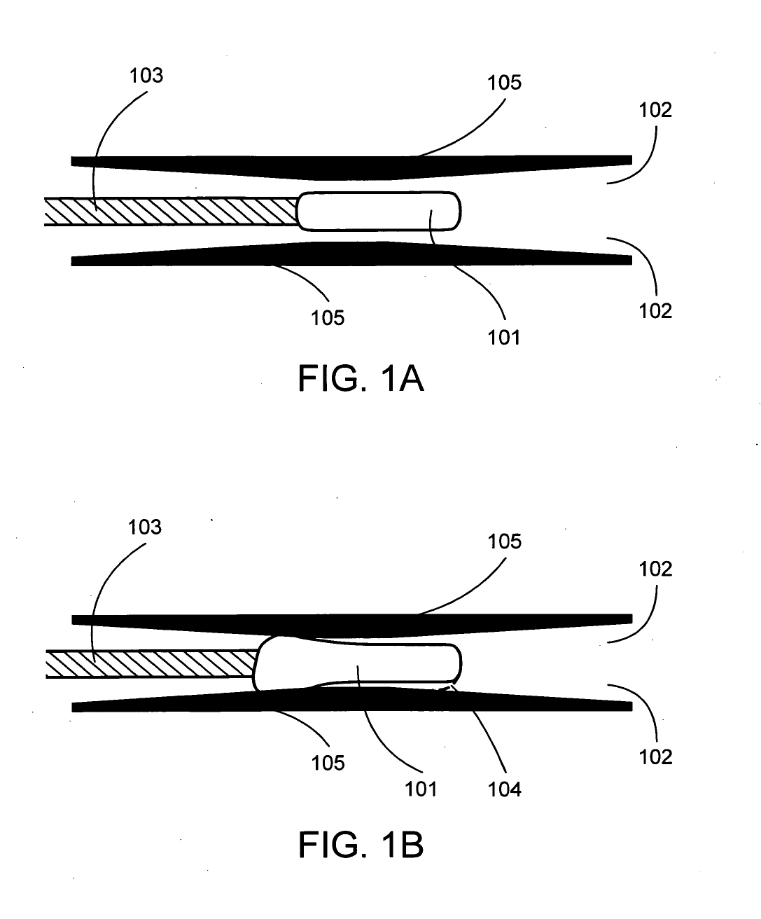

FIG. 1 depicts differential expansion that may be encountered with conventional elastomeric members. In FIG. 1A, a medical device 100 of the prior art comprising an elastomeric member 101 is delivered into an artery 102 within a patient containing unhealthy plaque 105 using a delivery means 103. For example, a conventional medical balloon is inserted into a patient using a catheter 104. After expansion, as seen in FIG. 1B, the balloon experiences differential expansion due to the balloon's construction. Differential expansion is detrimental because the balloon lacks uniformity. For example, the portion of the balloon depicted on the left side of FIG. 1B is closer to the walls of the artery 102 than the left side of the balloon. Differential expansion hinders efficacy of treatment because, for example, the unhealthy plaque 105 on right side of the artery is not receiving as much blockage-removing force as is the unhealthy plaque 105 on the left side of the artery. In FIG. 1B, the desired expansion profile 104 is indicated with a dotted line.

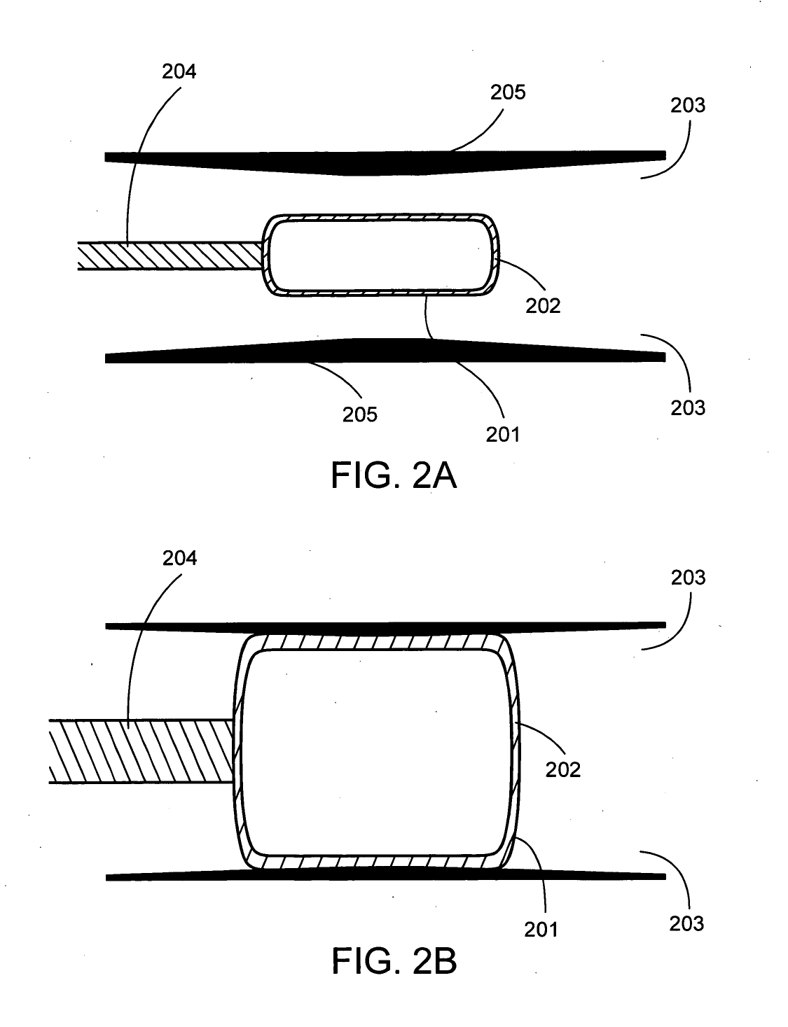

FIG. 2 depicts a medical device 200 in accordance with an embodiment of the invention, comprising an elastomeric member 201 in contact with at least one fiber oriented in two directions 202 located inside the elastomeric member. In FIG. 2A, the elastomeric member 201 is delivered to an artery 203 within a patient containing unhealthy plaque 205 using a catheter 204. For example, the elastomeric member 201 may be a balloon, having dimensions of approximately 6 centimeters in diameter, made of polyurethane and/or silicone and the fiber 202 may be made of lycra. After expansion, as seen in FIG. 2B, the balloon experiences desirable non-differential expansion due to fiber 202. As a result, all areas of the artery 203 receive equal blockage-removing force, enhancing the therapeutic value of the procedure.

Fibers employed in the invention that contact the elastomeric members form a pattern and are oriented in at least two directions within the pattern. The pattern is designed such that non-differential expansion is facilitated. Patterns may be regular or irregular, simple or complex, and single or multilayer, depending on the therapeutic application. Patterns include at least one opening or void between or among the fiber employed. The dimensions of the openings or voids ranges in size depending on the therapeutic application. Examples of patterns include, for example, grids, honeycombs, cocentric circles running in one or more directions, ellipsoids, and spirals.

The fibers employed in the invention may be made from any biocompatible material suitable for the therapy being administered with the elastomeric member. Such fibers include, for example, those made from catgut, chromic catgut, gore-tex, lycra, nylon, polyester, polypropylene, silk, stainless steel, nitinol, vicryl, or other suitable polymer, metal, or natural material. With regard to fiber density, the fibers may have a thickness, for example, of from about 0.001 to about 0.010 inches, with one or more wraps per inch. Fibers may be, for example, round, flat (including ribbon-like), and triangular. Selection of the fibers employed by one skilled in the art depends on the nature of the therapy. For example, in brachytherapy fibers are selected based on a number of criteria, including their ability to resist degradation by radiation. Generally, the fibers are selected so that their strength is greater than that of the elastomeric member itself. Generally, fibers with good tensile strength are selected so that the maximum dimensions of the elastomeric member-fibers complex is fixed.

The fibers may be located inside of, outside of, and/or within the elastomeric member. Whether the fibers are located inside of, outside of, or within the elastomeric member, the fibers define the maximum dimensions of the member.

For example, when the fiber is located inside of the elastomeric member, such as in FIG. 2, the elastomeric member has a framework over which it will conform.

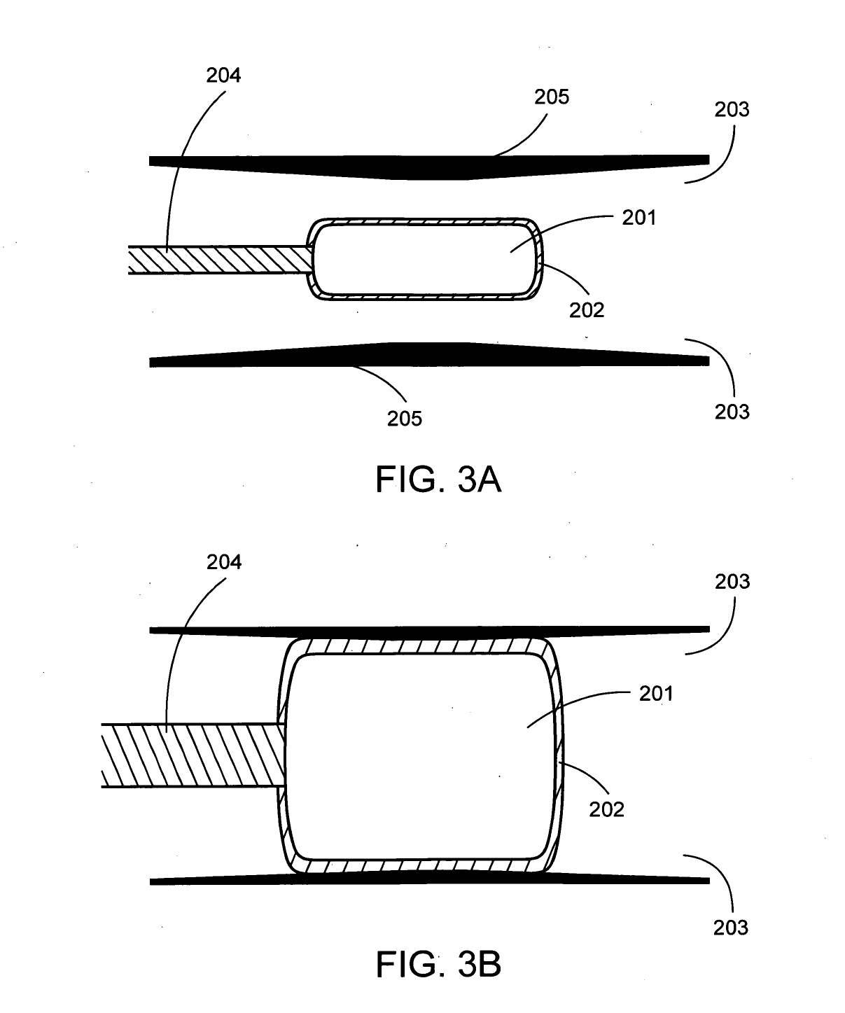

FIG. 3 depicts a medical device 200 in accordance with an embodiment of the invention, comprising an elastomeric member 201 in contact with at least one fiber oriented in two directions 202 located outside the elastomeric member. In FIG. 3A, the elastomeric member 201 is delivered to an artery 203 within a patient containing unhealthy plaque 205 using a catheter 204. After expansion, as seen in FIG. 3B, the balloon experiences desirable non-differential expansion due to the fiber 202. When the fiber is located inside of the elastomeric member, such as in the embodiment depicted in FIG. 3, the elastomeric member is unable to expand past the dimensions of the fiber. As a result, all areas of the artery 203 receive equal blockage-removing force, enhancing the therapeutic value of the procedure.

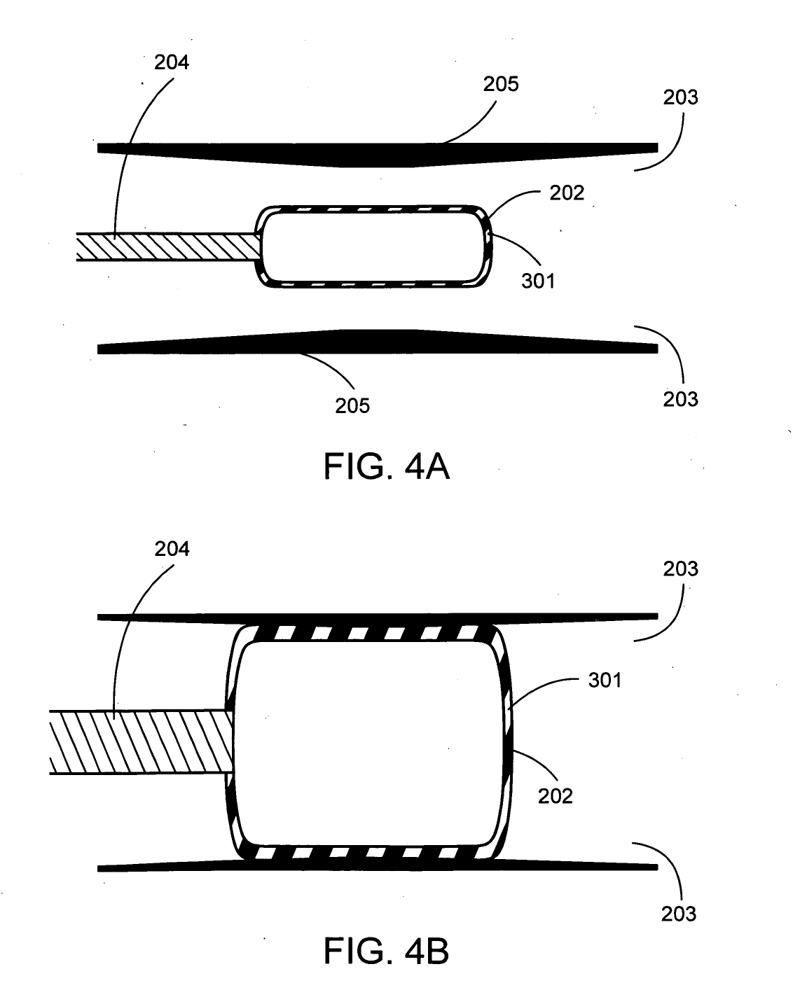

FIG. 4 depicts a medical device 200 in accordance with an embodiment of the invention, comprising an elastomeric member 301 in contact with at least one fiber oriented in two directions 202 located within the elastomeric member. In FIG. 4A, the elastomeric member 301 is delivered to an artery 203 within a patient containing unhealthy plaque 205 using a catheter 204. After expansion, as seen in FIG. 4B, the balloon experiences desirable non-differential expansion due to the fiber 202. When fiber is located within the elastomeric member, such as in FIG. 4, the elastomeric member is unable to expand independent of the dimensions of the fiber. As a result, all areas of the artery 203 receive equal blockage-removing force, enhancing the therapeutic value of the procedure.

The fibers employed in the invention may be oriented, for example, in at least two directions, including perpendicular directions. Those skilled in the art will appreciate that the orientation of the fibers will be designed to facilitate obtaining non-differential expansion.

While in no way limiting, the elastomeric members of the invention may comprise biocompatible, radiation-resistant polymers, such as Silastic rubbers, polyurethanes, polyethylene, polypropylene, polyester, PVC, and C-Flex.

In any embodiment, the orientation of fibers employed in the invention will depend on the specific application and device configuration. By way of example, the embodiments have fibers running in from at least one direction to up to multiple directions.

As used herein, the term “brachytherapy” refers to radiation therapy delivered by a spatially-confined source of therapeutic radiation. Often, the therapeutic radiation is administered within a patient's body, often at or near a tumor or other proliferative tissue disease site. Brachytherapy devices treat proliferative tissue disorders, such as cancerous tumors, by delivering radiation to the target area which contains both cancerous cells and healthy tissue. The radiation destroys the more radiosensitive cells, e.g., cancer cells, while hopefully minimizing damage to the surrounding healthy tissue. The most effective treatment delivers a dose above a minimum radiation dose necessary to destroy the proliferative tissue and below a maximum radiation dose to limit damage to healthy tissue. In addition to delivering a radiation dose within the proper range, brachytherapy devices may also deliver the radiation in a desired pattern. For example, it may be desirable to deliver radiation in a uniform three dimensional profile.

In use, the desired radiation dose is calculated based on factors such as the position of the radiation source, the type of radiation used, and the characteristics of the tissue and brachytherapy device. The brachytherapy device is then positioned within a tissue cavity and the dose is delivered. Unfortunately, variations in the brachytherapy device, in the surrounding tissue, or in the positioning of the radiation source can effect the delivered dose.

Some conventional brachytherapy devices include a catheter body member having a proximal end and a distal end, an inner spatial volume disposed proximate to the distal end of the catheter body member, an outer spatial volume defined by an expandable surface element disposed proximate to the distal end of the body member in a surrounding relation to the inner spatial volume, and a radiation source disposed in the inner spatial volume. The inner and outer spatial volumes are configured to provide an absorbed dose within a predetermined range throughout a target tissue. The target tissue is located between the outer spatial volume expandable surface and a minimum distance outward from the outer spatial volume expandable surface. The predetermined dose range is defined as being between a minimum prescribed absorbed dose for delivering therapeutic effects to tissue that may include cancer cells, and a maximum prescribed absorbed dose above which healthy tissue necrosis may result.

In other conventional brachytherapy devices of the prior art, the catheter body member may have a solid spherical radiation emitting material within a spatial volume. The device has a distal end, an inflation port, and a proximal end. For example, radioactive micro spheres of the type available from the 3M Company of St. Paul, Minn., may be used. This radioactive source is either preloaded into the catheter at the time of manufacture or loaded into the device after it has been implanted into the space formerly occupied by the excised tumor. For example, the solid radiation emitting material is inserted through catheter on a wire, using an afterloader. Such a solid radioactive core configuration offers an advantage in that it allows a wider range of radionuclides than if one is limited to liquids. Solid radionuclides that could be used with such a delivery device are currently generally available as brachytherapy radiation sources. However, such an apparatus can experience detrimental differential expansion.

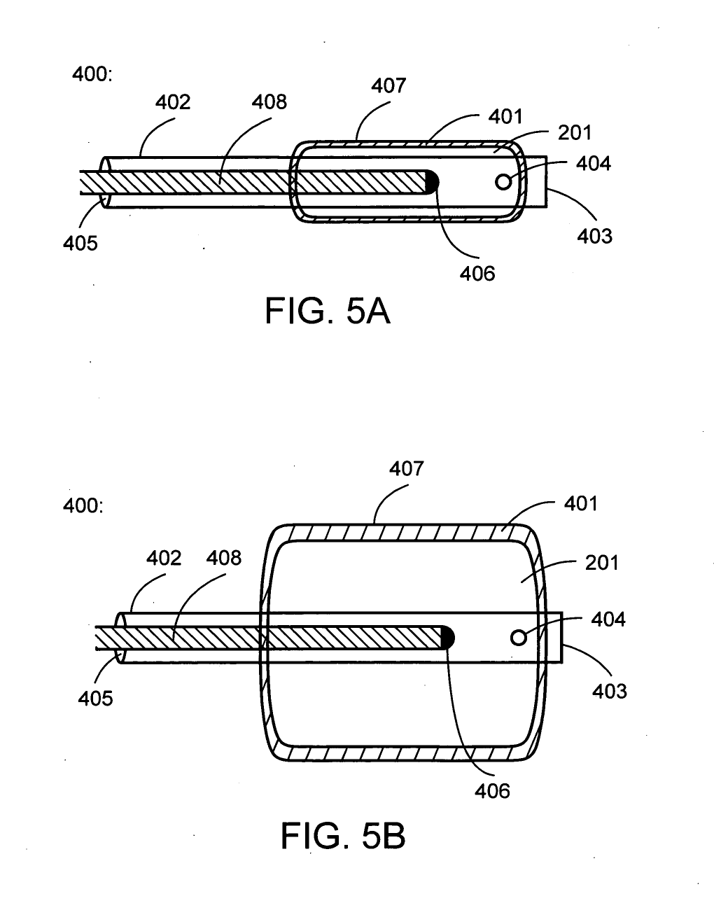

FIG. 5 depicts a brachytherapy apparatus 400 according to an embodiment of the present invention for delivering radioactive emissions to mammalian tissue wherein a radiation emitting source is contained within a spatial volume. In FIG. 5A, the brachytherapy apparatus 400 has an elastomeric member 201 in contact with at least one fiber 401, depicted prior to expansion. The brachytherapy apparatus 400 comprises a catheter 402, comprising a distal end 403, an inflation port 404, and a proximal end 405. The apparatus further comprises a radiation emitting material 406, which may be inserted through catheter 402 on a wire 408, within a spatial volume 407. The spatial volume 407 is defined by an elastomeric member 201 and at least one fiber 401 located outside the elastomeric member 201. In FIG. 5B, the brachytherapy apparatus of FIG. 5A is depicted following expansion, using the inflation port 404, of the expandable member 201.

The catheter 402 of the brachytherapy apparatus 400 depicted in FIG. 5 provides a means for positioning the expandable member 201 within a tissue cavity and presents a path for delivering radiation emitting material and inflation material, if used. Although the exemplary catheter depicted in FIG. 5 has a tubular construction, one of skill in the art readily appreciates that the catheter 402 may have a variety of shapes and sizes. Catheters suitable for use in the invention include catheters which are known in the art. Although catheters may be constructed from a variety of materials, in one embodiment the catheter material is silicone, for example a silicone that is at least partially radio-opaque, thus facilitating x-ray localization of catheter after insertion. Catheters may also include conventional adapters for attachment to a treatment fluid receptacle and the balloon, as well as devices, e.g., right-angle devices, for conforming the catheter to contours of the patient's body.

Considering that brachytherapy seeks to deliver a predetermined radiation dosing profile solely to target tissue so that target tissue is treated and healthy tissue is not damaged, non-differential expansion is critical to safe and effective therapy. If the radiation source is not centered within, for example, the medical balloon, a predetermined asymmetric radiation dosing profile may be employed to protect sensitive tissues, such as skin and the chest wall. Alternatively, therapy may be designed to deliver a non-uniform dose of radiation, due to offset of, for example, the medical balloon within the cavity. In either case, differential expansion of the balloon may result in the target tissue receiving too little radiation and healthy, non-target tissue receiving deleterious radiation. Differential expansion may also require the care giver to repeat the procedure(s) in an effort to obtain non-differential expansion. The elastomeric members of the present invention curb and/or prevent differential expansion and help maintain integrity of treatment planning profiles by preventing the need for recalculation and/or need to reposition the balloon, which can be painful to the patient and which can increase the risk of infection.

An advantage of the brachytherapy apparatus of the present invention is that it provides for treatment of tissue surrounding a cavity left by surgical removal of a tumor in a living patient. Because the elastomeric members of the brachytherapy apparatus of the present invention may by intraoperatively placed in the cavity formerly occupied by the tumor, a means for subsequent treatment of any residual tumor and/or infiltrating tumor cells is provided, without having to make additional surgical incisions. Yet another advantage of the elastomeric members of the present invention is their natural compliance to conform to the outline of the cavity to be treated, allowing for close approximation of the member to the treatment site.

The brachytherapy apparatus of the invention can be used in the treatment of a variety of malignant tumors, and is especially useful for in the treatment of brain and breast tumors.

Many breast cancer patients are candidates for breast conservation surgery, also known as lumpectomy, a procedure that is generally performed on early stage, smaller tumors. Breast conservation surgery is typically followed by postoperative radiation therapy. Studies report that 80% of breast cancer recurrences after conservation surgery occur near the original tumor site, strongly suggesting that a tumor bed “boost” of local radiation to administer a strong direct dose may be effective in killing any remaining cancer and preventing recurrence at the original site. The apparatus described herein can be used for either the primary or boost therapy. Numerous studies and clinical trials have established equivalence of survival for appropriate patients treated with conservation surgery plus radiation therapy compared to mastectomy.

Surgery and radiation therapy are also the standard treatments for malignant solid brain tumors. The goal of surgery is to remove as much of the tumor as possible without damaging vital brain tissue. The ability to remove the entire malignant tumor is limited by its tendency to infiltrate adjacent normal tissue. Partial removal reduces the amount of tumor to be treated by radiation therapy and, under some circumstances, helps to relieve symptoms by reducing pressure on the brain.

A method according to the invention for treating these and other malignancies begins by surgical resection of a tumor site to remove at least a portion of the cancerous tumor and create a resection cavity. Following tumor resection, but prior to closing the surgical site, the surgeon intra-operatively places a brachytherapy apparatus comprising an elastomeric member in contact with at least one fiber as described herein, but without having the radioactive source material loaded, into the tumor resection cavity. Once the patient has sufficiently recovered from the surgery, the brachytherapy apparatus is loaded with a radiation emitting source. The radioactive source dwells in the catheter until the prescribed dose of radiotherapy is delivered, typically for approximately a week or less. The radiation source is then retrieved and the catheter is removed. The radiation treatment may end upon removal of the brachytherapy apparatus, or the brachytherapy may be supplemented by further doses of radiation supplied externally.

Radiation emitting sources useful for the present invention include any radiation source which can deliver radiation to treat proliferative disorders, including high-dose radiation, medium-dose radiation, low-dose radiation, pulsed-dose radiation, external beam radiation, and combinations thereof. Such sources include predetermined radionuclides, for example, I-125, I-131, Yb-169, as well as other sources of radiation, such as radionuclides that emit photons, beta particles, or other therapeutic rays. Radiation emitting sources useful for the present invention may operate alone, or may be used in conjunction with radioactive ray absorbent material, such as air, water, and/or contrast materials. Radiation emitting sources useful for the present invention may include a single solid sphere, or may comprise a plurality of radioactive particles strategically placed so as to radiate in one or more directions with equal or varying intensities.

The radiation emitting source may also be a radioactive fluid made from any solution of radionuclide(s). Such a radioactive fluid may also be produced using a slurry of suitable fluid containing small particles of solid radionuclides, such as Au-198 and Y-90. Radionuclides may also be embodied in a gel.

By employing an elastomeric member in contact with at least one fiber of the present invention, differential expansion is greatly decreased, thereby resulting in more precise therapy. Similarly, brachytherapy apparatus of the present invention employing an elastomeric member in contact with at least one fiber of the present invention offers brachytherapy with greater precision.

A person skilled in the art will appreciate the foregoing as only illustrative of the principles of the invention, and that various modifications can be made by those skilled in the art without departing from the scope and spirit of the invention.

What is claimed is:

1. An expandable device for placement within a patient, comprising: an elastomeric member and at least one fiber in contact with said elastomeric member, wherein said fiber forms a pattern and is oriented in at least two directions within said pattern.

2. The expandable device of claim 1, wherein said elastomeric member has a round, square, rectangular, or oval profile configuration.

3. The expandable device of claim 1, wherein said expandable device comprises a plurality of fibers.

4. The expandable device of claim 1, wherein said pattern is regularly-spaced.

5. The expandable device of claim 1, wherein said at least one fiber is located inside of, outside of, or within said elastomeric member.

6. The expandable device of claim 1, wherein said expandable device comprises a plurality of regularly-spaced fibers in contact with said elastomeric member and running in perpendicular directions.

7. A brachytherapy apparatus for delivering radioactive emissions to mammalian tissue, comprising:
(a) an elastomeric member for placement within a patient and at least one fiber in contact with said elastomeric member;
(b) a catheter having a proximal end, a distal end, and a spatial volume at said distal end, wherein said spatial volume is defined by said elastomeric member; and
(c) a radiation source position disposed in said spatial volume.

8. The brachytherapy apparatus of claim 7, wherein said at least one fiber forms a pattern and is oriented in at least two directions within said pattern.

9. The brachytherapy apparatus of claim 7, wherein said elastomeric member has a round, square, rectangular, or oval profile configuration.

10. The brachytherapy apparatus of claim 7, wherein said expandable device comprises a plurality of fibers.

11. The brachytherapy apparatus of claim 7, wherein said at least one fiber forms a regularly-spaced pattern and is oriented in at least two directions within said pattern.

12. The brachytherapy apparatus of claim 7, wherein said at least one fiber is located inside of, outside of, or within said elastomeric member.

13. The brachytherapy apparatus of claim 7, wherein said expandable device comprises a plurality of regularly-spaced fibers in contact with said elastomeric member and running in perpendicular directions.

14. A method for performing a brachytherapy procedure on a patient, comprising:
inserting into the patient in need of brachytherapy a catheter having a proximal end, a distal end, and a spatial volume at said distal end, wherein said spatial volume is defined by an elastomeric member and at least one fiber in contact with said elastomeric member;
inflating or expanding said elastomeric member;
inserting a radiation source in said spatial volume of said catheter; and
removing said radiation source and said catheter from said patient.

15. The method of claim 14, wherein said at least one fiber forms a pattern and is oriented in at least two directions within said pattern.

16. The method of claim 14, wherein said elastomeric member has a round, square, rectangular, or oval profile configuration.

17. The method of claim 14, wherein said expandable device comprises a plurality of fibers.

18. The method of claim 14, wherein said at least one fiber forms a regularly-spaced pattern and is oriented in at least two directions within said pattern.

19. The method of claim 14, wherein said at least one fiber is located inside of, outside of, or within said elastomeric member.

20. The method of claim 14, wherein said expandable device comprises a plurality of regularly-spaced fibers in contact with said elastomeric member and running in perpendicular directions.
